(19) United States
(12) Patent Application Publication
(10) Pub. No.: US 2026/0263797 A1
(43) Pub. Date: Sep. 10, 2026

(54) MEDICAL IMPLANT WITH INTEGRATED ELECTRICAL AND VIBRATIONAL STIMULATION

(71) Applicant: Cochlear Limited, Macquarie University, NSW (AU)

(72) Inventors: Koen Erik Van den Heuvel, Hove (BE); Floriaan Van Reusel, Mechelen (BE); Antonin Rambault, Mechelen (BE)

(21) Appl. No.: 19/119,248

(22) PCT Filed: Sep. 26, 2023
(86) PCT No.: PCT/IB2023/059475

§ 371 Date: Apr. 8, 2025

Related U.S. Application Data
(60) Provisional application No. 63/381,006, filed on Oct. 26, 2022.

(51) Int. Cl. A61N 1/36; A61N 1/05; A61N 1/375

(52) U.S. Cl. CPC A61N 1/36038; A61N 1/0541; A61N 1/3754

(57) ABSTRACT

An apparatus includes a housing sealing an internal region within the housing from an external region outside the housing. The housing is configured to be implanted on or within a recipient's body. The apparatus further includes at least one actuator at least partially within the housing. The at least one actuator is configured to generate mechanical vibrational signals. The at least one actuator includes a coupling portion configured to be in mechanical communication with a fixture implanted on or within a recipient's body and configured to transmit the mechanical vibrational signals to the recipient's body. The apparatus further includes circuitry within the housing and configured to generate electrical stimulation signals configured to be received by the recipient's body. The apparatus further includes a plurality of electrical conduits in electrical communication with the circuitry and extending from the internal region to the external region. The plurality of electrical conduits are arranged in at least one arc segment at least partially encircling the coupling portion of the at least one actuator.

### BACKGROUND

### Field

The present application relates generally to medical implants (e.g., implantable medical prostheses) having active components (e.g., transducers; actuators; microphones; sensors).

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a housing sealing an internal region within the housing from an external region outside the housing. The housing is configured to be implanted on or within a recipient's body. The apparatus further comprises at least one actuator at least partially within the housing. The at least one actuator is configured to generate mechanical vibrational signals. The at least one actuator comprises a coupling portion configured to be in mechanical communication with a fixture implanted on or within a recipient's body and configured to transmit the mechanical vibrational signals to the recipient's body. The apparatus further comprises circuitry within the housing and configured to generate electrical stimulation signals configured to be received by the recipient's body. The apparatus further comprises a plurality of electrical conduits in electrical communication with the circuitry and extending from the internal region to the external region. The plurality of electrical conduits are arranged in at least one arc segment at least partially encircling the coupling portion of the at least one actuator.

In another aspect disclosed herein, a method comprises applying oscillating electrical voltage signals to a planar piezoelectric element having a central portion and a peripheral portion spaced from the central portion. The central portion is in mechanical communication with an abutment affixed to a fixture implanted on or within a recipient's body. The peripheral portion is affixed to at least one mass. The piezoelectric element responds to the electric voltage signals by imparting oscillatory motion to the at least one mass to generate vibrations and to transmit the vibrations to the fixture via the abutment. The method further comprises applying electrical stimulation signals to the recipient's body via a plurality of electrical feedthroughs substantially surrounding the abutment.

In another aspect disclosed herein, an apparatus comprises an implantable stimulator comprising a plurality of signal conduits and configured to emit non-vibrational stimulation signals via the plurality of signal conduits to be received by a cochlea of the recipient's body. The non-vibrational stimulation signals are configured to evoke a first hearing percept by the recipient in a first acoustic frequency range. The apparatus further comprises an implantable actuator comprising an abutment. The actuator is configured to emit vibrational signals via the abutment. The vibrational signals are configured to be received by the cochlea of the recipient's body and to evoke a second hearing percept by the recipient in a second acoustic frequency range. The second acoustic frequency range is lower than the first acoustic frequency range. The abutment is at least partially encircled by the plurality of signal conduits

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 is a perspective view of an example fully implantable middle ear implant auditory prosthesis implanted in a recipient in accordance with certain implementations described herein;

FIG. 3 schematically illustrate a portion of another example transcutaneous bone conduction auditory prosthesis implanted in a recipient in accordance with certain implementations described herein;

FIGS. 4A and 4B schematically illustrate side and top cross-sectional views, respectively, of a portion of an example apparatus in accordance with certain implementations described herein; and

FIG. 5 is a flow diagram of an example method in accordance with certain implementations described herein.

### DETAILED DESCRIPTION

Certain implementations described herein provide an auditory prosthesis that generates both mechanical vibrational signals and electrical stimulation signals that are received by a cochlea of a recipient's body to evoke a hearing percept by the recipient. For example, the hearing percept evoked by the mechanical vibrational signals can supplement the hearing percept evoked by the electrical stimulation signals at low (e.g., bass) acoustic frequencies. The mechanical vibrational signals are generated by an actuator comprising a piezoelectric oscillator and are transmitted via an abutment and a fixture to the recipient's body. The electrical stimulation signals are generated by circuitry and transmitted via a plurality of electrical conduits that have a space-efficient arrangement in which the electrical conduits are part of one or more feedthroughs having a curved shape (e.g., banana-shaped) and extending at least partially around the abutment. In addition, the vibrating mass of the piezoelectric actuator can be positioned at or near an outer periphery of the piezoelectric oscillator to generally surround a region containing the circuitry.

The teachings detailed herein are applicable, in at least some implementations, to any type of implantable medical system utilizing an implantable transducer assembly configured to provide vibrational signals and stimulation signals (e.g., electrical stimulation signals; optical stimulation signals; electromagnetic stimulation signals) to the recipient's body in response to received information and/or control signals (e.g., implantable sensor prostheses; implantable stimulation system). For example, the implantable medical system can comprise an auditory prosthesis system configured to generate and apply stimulation signals (e.g., electrical and vibrational) that are perceived by the recipient as sounds (e.g., evoking a hearing percept). Such implantable transducer assemblies can include but are not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices (e.g., auditory brain stimulators), and/or combinations or variations thereof, or any other suitable hearing prosthesis system with or without one or more external components. Merely for ease of description, apparatus and methods disclosed herein are primarily described with reference to an illustrative auditory prosthesis system, namely a combined cochlear implant and bone conduction device, but implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses.

The teachings detailed herein and/or variations thereof may also be used with a variety of other medical devices that provide a wide range of therapeutic benefits to recipients, patients, or other users. For example, other sensory prosthesis systems that are configured to evoke other types of neural or sensory (e.g., sight, tactile, smell, taste) percepts are compatible with certain implementations described herein, including but are not limited to: vestibular devices (e.g., vestibular implants), visual devices (e.g., bionic eyes), visual prostheses (e.g., retinal implants), somatosensory implants, and chemosensory implants. In some implementations, the teachings detailed herein and/or variations thereof can be utilized in other types of implantable medical devices beyond sensory prostheses. For example, apparatus and methods disclosed herein and/or variations thereof can be used with one or more of the following: sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. Implementations can include any type of medical system that can utilize the teachings detailed herein and/or variations thereof (e.g., systems that may benefit from having an adjustable orientation of at least a portion of the implanted device during implantation).

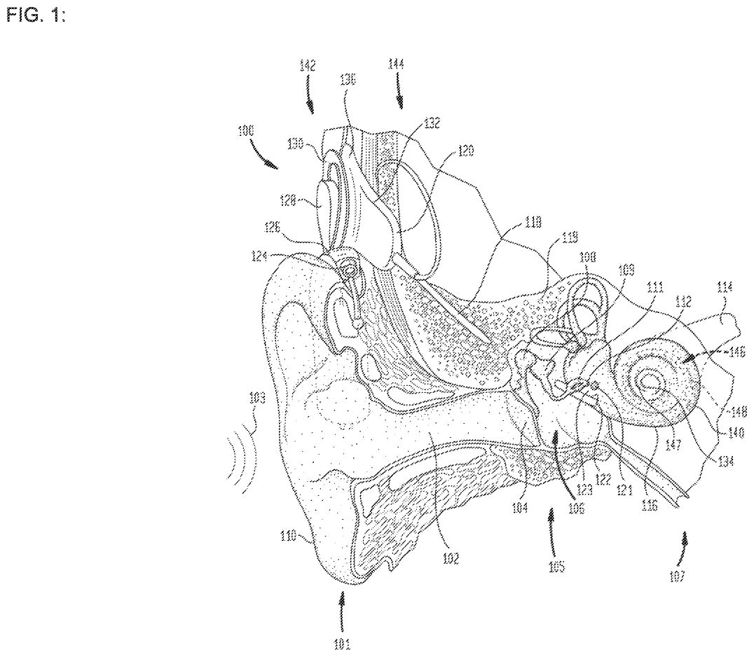

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1 with a subcutaneously implantable microphone assembly, as described more fully herein. In certain implementations, the example cochlear implant auditory prosthesis 100 of FIG. 1 can be in conjunction with a reservoir of liquid medicament as described herein.

As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

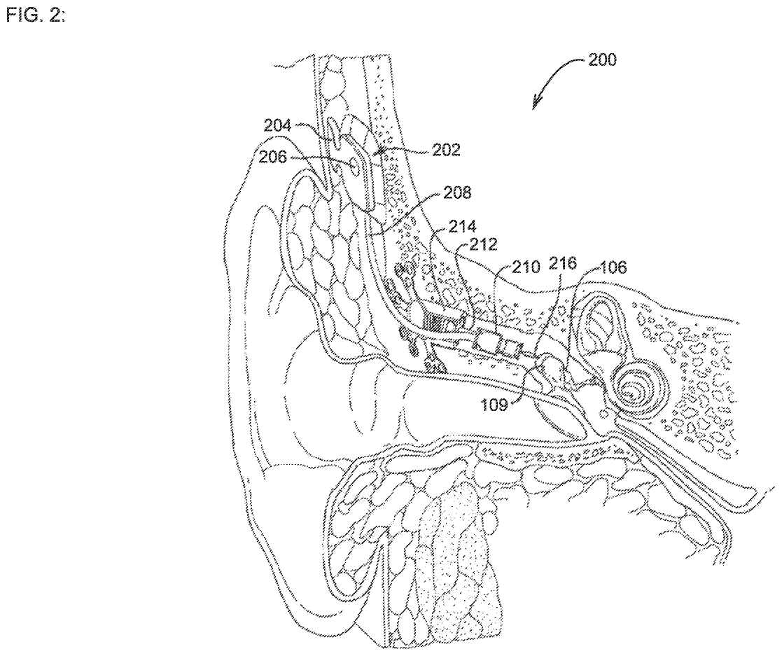

FIG. 2 schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 2 comprises a biocompatible implantable assembly 202 (e.g., comprising an implantable capsule) located subcutaneously (e.g., beneath the recipient's skin and on a recipient's skull). While FIG. 2 schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 2, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1 and 2 can comprise an implantable microphone assembly, such as the microphone assembly 206 shown in FIG. 2. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 2 is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

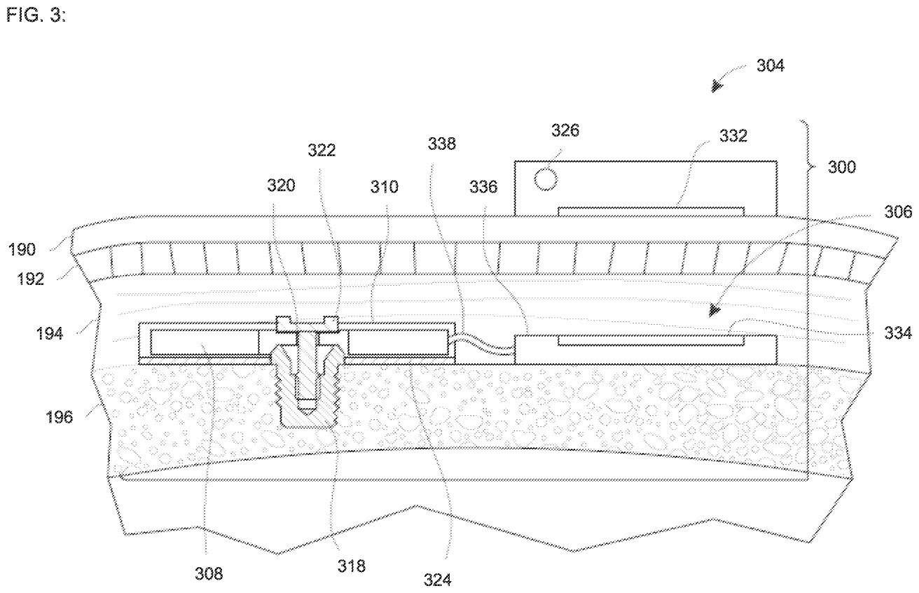

FIG. 3 schematically illustrate a portion of an example transcutaneous bone conduction auditory prosthesis 300 implanted in a recipient in accordance with certain implementations described herein. As schematically illustrated by FIG. 3, the example transcutaneous bone conduction auditory prosthesis 300 comprises an external device 304 and an implantable component 306. The auditory prosthesis 300 is an active transcutaneous bone conduction auditory prosthesis in that the vibrating actuator 308 is located in the implantable component 306. For example, a vibratory element in the form of a vibrating actuator 308 is located in a housing 310 of the implantable component 306. In certain implementations, the vibrating actuator 308 is a device that converts electrical signals into vibration. The vibrating actuator 308 can be in direct contact with the outer surface of the recipient's bone 196 (e.g., the vibrating actuator 308 is in substantial contact with the recipient's bone 196 such that vibration forces from the vibrating actuator 308 are communicated from the vibrating actuator 308 to the recipient's bone 196). In certain implementations, there can be one or more thin non-bone tissue layers (e.g., a silicone layer 324) between the vibrating actuator 308 and the recipient's bone 196 (e.g., bone tissue; skull bone) while still permitting sufficient support so as to allow efficient communication of the vibration forces generated by the vibrating actuator 308 to the recipient's bone 196.

In certain implementations, the external component 304 includes a sound input element 326 that converts sound into electrical signals. Specifically, the auditory prosthesis 300 provides these electrical signals to the vibrating actuator 308, or to a sound processor (not shown) that processes the electrical signals, and then provides those processed signals to the implantable component 306 through the tissue of the recipient (e.g., skin 190, fat 192, muscle 194) via a magnetic inductance link. For example, a communication coil 332 of the external component 304 can transmit these signals to an implanted communication coil 334 located in a housing 336 of the implantable component 306. Components (not shown) in the housing 336, such as, for example, a signal generator or an implanted sound processor, then generate electrical signals to be delivered to the vibrating actuator 308 via electrical lead assembly 338. The vibrating actuator 308 converts the electrical signals into vibrations. In certain implementations, the vibrating actuator 308 can be positioned with such proximity to the housing 336 that the electrical leads 338 are not present (e.g., the housing 310 and the housing 336 are the same single housing containing the vibrating actuator 308, the communication coil 334, and other components, such as, for example, a signal generator or a sound processor).

In certain implementations, the vibrating actuator 308 is mechanically coupled to the housing 310. The housing 310 and the vibrating actuator 308 collectively form a vibrating element. The housing 310 can be substantially rigidly attached to a bone fixture 318.

In this regard, the housing 310 can include a through hole 320 that is contoured to the outer contours of the bone fixture 318. The screw 322 can be used to secure the housing 310 to the bone fixture 318. As can be seen in FIG. 3, the head of the screw 322 is larger than the through hole 320 of the housing 310, and thus the screw 322 positively retains the housing 310 to the bone fixture 318. A portion of the screw 322 interfaces with the bone fixture 318, thus permitting the screw 322 to readily fit into an existing bone fixture 318 used in a percutaneous bone conduction device (or an existing passive bone conduction device). In certain implementations, the screw 322 is configured so that the same tools and procedures that are used to install and/or remove an abutment screw from the bone fixture 318 can be used to install and/or remove the screw 322 from the bone fixture 318.

The bone fixture 318 can be made of any material that has a known ability to integrate into surrounding bone tissue (e.g., comprising a material that exhibits acceptable osseointegration characteristics). In certain implementations, the bone fixture 318 is formed from a single piece of material (e.g., titanium) and comprises outer screw threads forming a male screw which is configured to be installed into the skull bone 196 and a flange configured to function as a stop when the fixture 318 is implanted into the skull bone 196. The screw threads can have a maximum diameter of about 3.5 mm to about 5.0 mm, and the flange can have a diameter which exceeds the maximum diameter of the screw threads (e.g., by approximately 10%-20%). The flange can have a planar bottom surface for resting against the outer bone surface, when the fixture 318 has been screwed down into the skull bone 196. The flange prevents the fixture 318 (e.g., the screw threads) from potentially completely penetrating completely through the bone 196.

The body of the fixture 318 can have a length sufficient to securely anchor the fixture 318 to the skull bone 196 without penetrating entirely through the skull bone 196. The length of the body can therefore depend on the thickness of the skull bone 196 at the implantation site. For example, the fixture 318 can have a length, measured from the planar bottom surface of the flange to the end of the distal region (e.g., the portion farthest from the flange), that is no greater than 5 mm or between about 3.0 mm to about 5.0 mm, which limits and/or prevents the possibility that the fixture 318 might go completely through the skull bone 196. The interior of the fixture 318 can further include an inner lower bore having female screw threads configured to mate with male screw threads of the screw 320 to the fixture 318. The fixture 318 can further include an inner upper bore that receives a bottom portion of the abutment 312.

The example auditory prostheses 100 shown in FIG. 1 utilizes an external microphone 124, the auditory prosthesis 200 shown in FIG. 2 utilizes an implantable microphone assembly 206 comprising a subcutaneously implantable acoustic transducer, and the example transcutaneous bone conduction auditory prosthesis 300 of FIG. 3 comprises an external sound input element 326 (e.g., external microphone). In certain implementations described herein, a subcutaneously implantable sound input assembly (e.g., implanted microphone) is used with the auditory prostheses 100, 200, 300 and/or one or more external microphone assemblies is used with the auditory prostheses 100, 200, 300. In certain implementations, an external microphone assembly can be used to supplement an implantable microphone assembly of the auditory prosthesis 100, 200, 300. Thus, the teachings detailed herein and/or variations thereof can be utilized with any type of external or implantable microphone arrangement, and the acoustic prostheses 100, 200, 300 shown in FIGS. 1, 2, and 3 are merely illustrative.

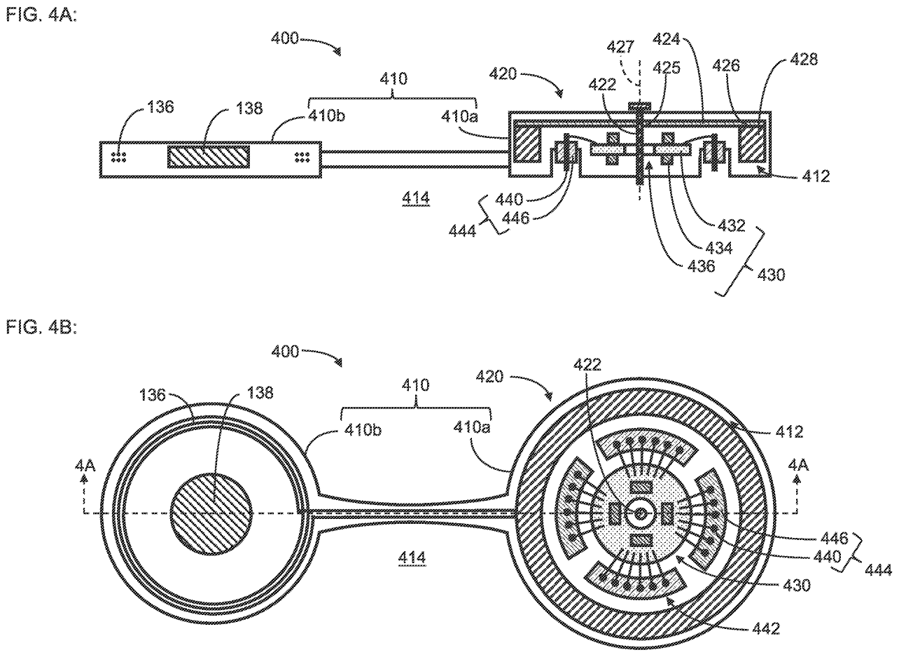

FIGS. 4A and 4B schematically illustrate side and top cross-sectional views, respectively, of a portion of an example apparatus 400 in accordance with certain implementations described herein. The apparatus 400 comprises a housing 410 sealing an internal region 412 within the housing 410 from an external region 414 outside the housing 410. The housing 410 is configured to be implanted on or within a recipient's body. The apparatus 400 further comprises at least one actuator 420 at least partially within the housing 410. The at least one actuator 420 is configured to generate mechanical vibrational signals. The at least one actuator 420 comprises a coupling portion 422 configured to be in mechanical communication with a fixture (e.g., fixture 318) implanted on or within a recipient's body and configured to transmit the mechanical vibrational signals to the recipient's body. The apparatus 400 further comprises circuitry 430 within the housing 410 and configured to generate electrical stimulation signals configured to be received by the recipient's body. The apparatus 400 further comprises a plurality of electrical conduits 440 in electrical communication with the circuitry 430 and extending from the internal region 412 to the external region 414. The plurality of electrical conduits 440 are arranged in at least one arc segment 442 at least partially encircling the coupling portion 422 of the at least one actuator 420.

In certain implementations, the housing 410 is configured to hermetically seal the at least one actuator 420 (e.g., within the internal region 412) from an environment (e.g., the external region 414) surrounding the at least one actuator 420. The housing 410 can have a length and/or a width less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters), and/or a thickness less than or equal to 7 millimeters (e.g., in a range of less than or equal to 6 millimeters, in a range of less than or equal to 5 millimeters; in a range of less than or equal to 4 millimeters). The housing 410 of certain implementations comprises at least one biocompatible material (e.g., plastic; PEEK; silicone; ceramic; zirconium oxide).

In certain implementations, the housing 410 comprises a single (e.g., unitary) housing while in certain other implementations, the housing 410 comprises multiple housing portions spaced from but in operable communication with one another and containing different portions of the apparatus 400. For example, as schematically illustrated by FIGS. 4A-4B, the housing 410 comprises a first housing portion 410a and a second housing portion 410b, the first housing portion 410a containing (e.g., hermetically sealing) the at least one actuator 420 and at least a portion of the circuitry 430, and the second housing portion 410b containing (e.g., hermetically sealing) an internal receiver unit 132 in electrical communication with the circuitry 430. For example, the internal receiver unit 132 can comprise an internal coil 136 and a magnet 138 fixed relative to the internal coil 136, the magnet 138 configured to hold an external device on the recipient's body over the tissue overlying the second housing portion 410b. The internal coil 136 can be configured to receive power and/or data signals from an external coil of the external device via a transcutaneous energy transfer link (e.g., an inductive RF link).

In certain implementations, the at least one actuator 420 is configured to generate vibrational energy (e.g., vibrations) within a range of vibrational frequencies that are perceptible by the recipient as sound (e.g., a range of 20 Hz to 20 kHz), which are referred to herein as auditory vibrations. The coupling portion 422 is part of a propagation path for the auditory vibrations to be transmitted to the recipient's body (e.g., via fixture 318) and to propagate via bone conduction to an inner ear region (e.g., within the temporal bone 196 and comprising the vestibule, the cochlea 140, and the semicircular canals) and/or a middle ear region (e.g., within the recipient's head, partially bounded by the tympanic membrane 104 and comprising the ossicles 106, the round window 121, the oval window 112, and the Eustachian tube) to be detected as sound.

In certain implementations, the at least one actuator 420 comprises at least one piezoelectric component 424 and at least one mass 428. The at least one piezoelectric component 424 is configured to undergo bending oscillations in response to received electric voltage signals (e.g., from the circuitry 430) and these bending oscillations move the at least one mass 428. As schematically illustrated by FIGS. 4A and 4B, the at least one piezoelectric component 424 of certain implementations comprises a first portion 425 in mechanical communication with the coupling portion 422 and a second portion 426 spaced from the first portion 425, the second portion 426 in mechanical communication with the at least one mass 428.

In certain implementations, the at least one piezoelectric component 424 comprises a unitary (e.g., single; monolithic) component comprising at least one piezoelectric material. The at least one piezoelectric component 424 of certain implementations comprises two or more layers in mechanical communication with one another (e.g., bonded together) into a unitary component (e.g., a stack), at least one of the layers comprising at least one piezoelectric material (e.g., unimorph having one piezoelectric layer and a non-piezoelectric layer; bimorph having two or more piezoelectric layers). The unitary component can comprise other non-piezoelectric materials, such as a bonding material (e.g., adhesive; epoxy; metal) between piezoelectric layers, electrically conductive material (e.g., metal) configured to apply electrical voltage signals to the at least one piezoelectric material, and/or a non-piezoelectric layer (e.g., metal backplate) affixed to the at least one piezoelectric material. In certain implementations, the number of layers of the at least one piezoelectric component 424 are selected to provide a predetermined power, size (e.g., area, thickness), stiffness, and/or resonance frequency. Examples of piezoelectric materials compatible with certain implementations described herein include but are not limited to: quartz; gallium orthophosphate; langasite; barium titanate; lead titanate; lead zirconate titanate (PZT); potassium niobate; lithium niobate; lithium tantalate; sodium tungstate; sodium potassium niobate; bismuth ferrite; sodium niobate; polyvinylidene fluoride; macro fiber composite (MFC); other piezoelectric crystals, ceramics, or polymers.

In certain implementations, the at least one piezoelectric component 424 comprises a substantially planar piezoelectric slab (e.g., plate; sheet), the first portion 425 comprises a central portion of the piezoelectric slab substantially surrounding and in mechanical communication with the coupling portion 422, and the second portion 426 comprises a peripheral portion of the piezoelectric slab spaced from the coupling portion 422. In certain implementations, the piezoelectric slab is a generally rectangular plate or a generally circular disk (e.g., as schematically illustrated by FIGS. 4A-4B). Other planar shapes are also compatible with certain implementations described herein (e.g., oval; polygonal with 5, 6, 7, 8, or more sides; geometric; non-geometric; regular; irregular). In certain implementations, the piezoelectric slab has a length (e.g., in a range of 2 millimeters to 30 millimeters; in a range of 10 millimeters to 20 millimeters), a width substantially perpendicular to the length (.g., in a range of 2 millimeters to 30 millimeters; in a range of 10 millimeters to 20 millimeters), and a thickness substantially perpendicular to the length and to the width (e.g., in a range of less than 2 millimeters; less than 1 millimeter; greater than 300 microns). Various configurations and geometries of the piezoelectric slab are compatible with certain implementations described herein (see, e.g., “Piezoelectric Ceramic Products: Fundamentals, Characteristics and Applications,” Physik Instruments (PI) GmbH & Co., Lederhose, Germany, www. piceramic. com, (2016)).

In certain implementations, the first portion 425 of the at least one piezoelectric component 424 is affixed to the coupling portion 422 (e.g., via a clamp, screw, adhesive, or other coupler) and does not substantially move relative to the coupling portion 422 during the bending oscillations of the at least one piezoelectric component 424. For example, the first portion 425 can comprise a hole (e.g., the hole has an inner perimeter that is part of the first portion 425) with the coupling portion 422 extending from the fixture along a longitudinal axis 427, the at least one piezoelectric slab extending along a plane substantially perpendicular to the longitudinal axis 427, and the coupling portion 422 extending through the hole and affixed to the surrounding first portion 425.

In certain implementations, the second portion 426 of the at least one piezoelectric component 424 is configured to substantially move relative to the coupling portion 422 during the bending oscillations of the at least one piezoelectric component 424 (e.g., in response to time-varying electrical voltage signals applied across portions of the at least one piezoelectric component 424). For example, the second portion 426 can comprise at least a portion of an outer perimeter of the piezoelectric slab that is in mechanical communication with the at least one mass 428 (e.g., via a clamp, screw, adhesive, or other coupler), such that the bending oscillations move the second portion 426 and the at least one mass 428 along a direction substantially parallel to the longitudinal axis 427 of the coupling portion 422 (e.g., substantially perpendicular to the piezoelectric slab).

In certain implementations, the at least one mass 428 comprises one or more materials having sufficiently large mass density and dimensions (e.g., length; width; thickness; volume) such that the at least one mass 428 has a mass (e.g., weight) configured to achieve a predetermined resonant frequency for the bending oscillations (e.g., the generated vibrations) (e.g., in a range of 60 Hz to 3 kHz; in a range of 250 Hz to 3 kHz; in a range of 60 Hz to 250 Hz). Examples of such materials of the at least one mass 428 include but are not limited to: tungsten; tungsten alloy; osmium; osmium alloy. The at least one mass 428 can comprise a unitary (e.g., single; monolithic) mass or component, multiple components (e.g., two or more sub-masses) that are affixed to one another, and/or multiple components that are separate from one another. In certain implementations, the at least one mass 428 comprises separate masses positioned at separate locations at the second portion 426 of the at least one piezoelectric component 424. For example, the at least one mass 428 can comprise two separate masses positioned at opposite ends of a substantially rectangular piezoelectric slab and spaced from the coupling portion 422. In certain other implementations, the at least one mass 428 extends substantially completely around an outer perimeter of the piezoelectric slab. For example, as schematically illustrated in FIGS. 4A-4B, the at least one mass 428 comprises a single mass (e.g., substantially circular; donut-shaped) encircling a hole (e.g., with an inner diameter and/or an inner perimeter) substantially surrounding the coupling portion 422, the circuitry 430, and the plurality of electrical conduits 440. The at least one mass 428 can be affixed to the outer perimeter of the substantially circular piezoelectric slab. The mass, inner diameter, and/or outer diameter of the at least one mass 428 can be sufficiently large to tune the predetermined resonant frequency of the at least one actuator 420 to be in the bass region (e.g., in a range of 60 Hz to 250 Hz).

In certain implementations, the at least one mass 428 at least partially bounds a region below the at least one piezoelectric component 424 and surrounding the coupling portion 422, the region configured to contain at least a portion of the circuitry 430. In certain implementations, as schematically illustrated in FIG. 4A, the at least one mass 428 is below the at least one piezoelectric component 424, while in certain other implementations, at least some of the at least one mass 428 is above the at least one piezoelectric component 424 (e.g., the at least one mass 428 is substantially evenly distributed above and below the at least one piezoelectric component 424). By having most of the at least one mass 428 below the at least one piezoelectric component 424 and/or having the region below the at least one piezoelectric component 424 containing at least a portion of the circuitry 430, certain implementations can provide an actuator 420 having a smaller height than conventional actuators.

In certain implementations, the circuitry 430 comprises a printed-circuit board 432 and a plurality of circuit elements 434 (e.g., one or more integrated circuits; one or more microcontrollers; one or more microprocessors; resistors; capacitors; inductors; electrical conduits) on and/or within the printed-circuit board 432 (e.g., affixed to a first side of the printed-circuit board 432 and/or to a second side of the printed-circuit board 432). As schematically illustrated by FIGS. 4A-4B, the printed-circuit board 432 of certain implementations has a hole 434 through which the coupling portion 422 extends. In certain implementations, the inner perimeter of the hole 434 is spaced from the coupling portion 422 such that the printed-circuit board 432 does not affect vibrations transmitted through the coupling portion 422 to the recipient's body.

In certain implementations, each electrical conduit 440 of the plurality of electrical conduits 440 comprises an electrically conductive material (e.g., gold; platinum; other metal and/or alloy), has a length in a range of 0.5 millimeter to 3 millimeters, and a thickness in a range of 100 microns to 500 microns. As schematically illustrated by FIGS. 4A-4B, the plurality of electrical conduits 440 are in electrical communication with the circuitry 430 and are configured to transmit electrical signals from the circuitry 430 within the inner region 412 to the external region 414. For example, the apparatus 200 can comprise at least one electrical feedthrough 444 comprising an electrically insulating portion 446 (e.g., plastic; polymer; ceramic) and one or more electrical conduits 440 extending through the electrically insulating portion 446. The at least one electrical feedthrough 444 can be a portion of the housing 410 and affixed to other portions of the housing 410 such that the internal region 412 is sealed from the external region 414. The electrically insulating portion 446 can have a thickness in a direction substantially parallel to the longitudinal axis 427 in a range of 0.5 millimeter to 5 millimeters and a width in a direction substantially perpendicular to the longitudinal axis 427 in a range of 1 millimeter to 5 millimeters. The electrically insulating portion 446 can extend around the longitudinal axis 427 with an angular extent in a range of 5 degrees to 360 degrees (e.g., in a range of 15 degrees to 80 degrees).

As schematically illustrated by FIGS. 4A-4B, in certain implementations, each of the at least one electrical feedthrough 444 has a shape of an arc segment of a circular annulus, while in certain other implementations, the at least one electrical feedthrough 444 has other shapes (e.g., segments of other curved and non-curved shapes; banana-shaped; bean-shaped; boomerang-shaped). As schematically illustrated by FIGS. 4A-4B, in certain implementations, each of the at least one electrical feedthrough 444 has substantially the same distance from the coupling portion 442, while in certain other implementations, the distances of the electrical feedthroughs 444 can differ from one another (e.g., two or more of the electrical feedthroughs 444 at different radial distances from the coupling portion 422). As schematically illustrated by FIGS. 4A-4B, in certain implementations, each of the electrical conduits 440 extending through an electrical feedthrough 444 has substantially the same distance from the coupling portion 422, while in certain other implementations, the distances of the electrical conduits 440 extending through the same electrical feedthrough 444 can differ from one another (e.g., two or more of the electrical conduits 440 at different distances from the coupling portion 422).

FIGS. 4A-4B schematically illustrate an example apparatus 400 having 24 electrical conduits 440 arranged in four arc segments 442 that at least partially encircle the coupling portion 422 of the at least one actuator 420, the electrical conduits 440 of each arc segment 442 extending through the electrical insulating portion 446 of a corresponding electrical feedthrough 444. Other numbers of electrical conduits 440 (e.g., in a range of 10 to 20; in a range of 20 to 30, in a range of 30 or more) and other numbers of arc segments 442 and/or electrical feedthroughs 444 (e.g., 1, 2, 3, 5, 6, or more) are also compatible with certain implementations described herein. In certain implementations (see, e.g., FIGS. 4A-4B), the electrical feedthroughs 444 are spaced from one another in an azimuthal direction around the coupling portion 422 (e.g., in certain implementations in which the portions of the housing 410 between adjacent electrical feedthroughs 444 provide more mechanical strength than would a single, fully circular electrical feedthrough 444).

FIGS. 4A-4B schematically illustrates each of the arc segments 442 having substantially the same distance from the coupling portion 422. In certain other implementations, two or more of the arc segments 442 have substantially different distances from the coupling portion 422. For example, a first arc segment 442 can have a first radial distance from the coupling portion 442 and a second arc segment 442 can have a second radial distance from the coupling portion 442, the second radial distance greater than the first radial distance. The first and second arc segments 442 can be segments of different circles that are concentric with one another.

FIGS. 4A-4B schematically illustrate an example apparatus 400 in which each electrical feedthrough 444 has six of the electrical conduits 440 extending therethrough. Other numbers of electrical conduits 440 per electrical feedthrough 444 extending therethrough (e.g., 1, 2, 3, 4, 5, or more) are also compatible with certain implementations described herein. While FIGS. 4A-4B schematically illustrate each electrical feedthrough 444 having substantially the same dimensions and having the same number of electrical conduits 440 extending therethrough, in certain other implementations, two or more of the electrical feedthroughs 444 have substantially different dimensions and/or numbers of electrical conduits 440 extending therethrough.

In certain implementations, the circuitry 430 is configured to generate electrical stimulation signals configured to be received by the recipient's body. In certain such implementations, the apparatus 200 further comprises a plurality of electrically conductive wires in the external region 414 and a plurality of stimulation electrodes in the external region 414 (e.g., an elongate electrode assembly 118; not shown in FIGS. 4A-4B). Each electrically conductive wire of the plurality of electrically conductive wires can have a first portion in electrical communication with a corresponding electrical conduit 440 of the plurality of electrical conduits 440 and a second portion in electrical communication with a corresponding stimulation electrode of the plurality of stimulation electrodes, such that the plurality of electrically conductive wires and the plurality of stimulation electrodes are configured to transmit the electrical stimulation signals from the plurality of electrical conduits 440 to the recipient's body. For example, the circuitry 430 can comprise a stimulator unit 120 of a cochlear implant auditory prosthesis 100 and the electrical stimulation signals can be configured to be transmitted from the circuitry 430 and received by a cochlea 140 of the recipient's body via an elongate electrode assembly 118 (e.g., the plurality of electrically conductive wires and the plurality of stimulation electrodes). The electrical stimulation signals can be configured to evoke a first hearing percept by the recipient in a first acoustic frequency range (e.g., in a range of 250 Hz to 3 kHz).

In certain implementations, the circuitry 430 is further configured to control the at least one actuator (e.g., the generation of the mechanical vibrational signals). For example, the mechanical vibrational signals can be configured to be received by the cochlea 140 of the recipient's body (e.g., the same cochlea 140 that receives the electrical stimulation signals via the plurality of electrical conduits 440) to evoke a second hearing percept by the recipient in a second acoustic frequency range (e.g., in a bass range; e.g., in a range of 60 Hz to 250 Hz), the second acoustic frequency range lower than the first acoustic frequency range. Thus, the apparatus 400 of certain implementations is configured to provide both electrical stimulation signals (e.g., of a cochlear implant auditory prosthesis 100) and mechanical vibrational signals (e.g., of a fully implantable auditory prosthesis 200; of a bone conduction auditory prosthesis 300). In certain implementations, besides providing a transmission path for the mechanical vibration signals from the apparatus 400 to the recipient's body, the coupling portion 422 is electrically conductive and is in electrical communication with the circuitry 430 and the recipient's body (e.g., via the fixture 318) and is configured to be a reference electrode for the electrical stimulation signals.

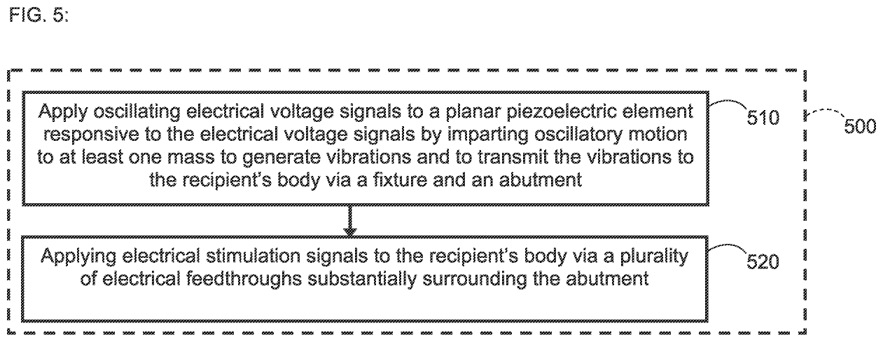

FIG. 5 is a flow diagram of an example method 500 in accordance with certain implementations described herein. While the example method 500 is described herein by referring to the example apparatus 400 of FIGS. 4A-4B, other apparatuses are also compatible with the example method 500 in accordance with certain implementations described herein. For example, the method 500 described herein can be applied to any of a variety of implantable medical devices.

In an operational block 510, the method 500 comprises applying oscillating electric voltage signals to a planar piezoelectric element (e.g., piezoelectric component 424) having a central portion (e.g., first portion 425) and a peripheral portion (e.g., second portion 426) spaced from the central portion. The central portion is in mechanical communication with an abutment (e.g., coupling portion 422) affixed to a fixture (e.g., fixture 318) implanted on or within a recipient's body. The peripheral portion is affixed to at least one mass (e.g., mass 428), and the piezoelectric element is responsive to the electric voltage signals by imparting oscillatory motion to the at least one mass to generate vibrations and to transmit the vibrations to the recipient's body (e.g., via the fixture and via the abutment).

In an operational block 520, the method 500 further comprises applying electrical stimulation signals to the recipient's body via a plurality of electrical feedthroughs substantially surrounding the abutment. For example, the electrical feedthroughs 444 can each comprise an electrically insulating portion 446 and at least one electrical conduit 440 extending from a first side of the electrically insulating portion 446 (e.g., the first side at least partially bounding the internal region 412 within the housing 410) to a second side of the electrically insulating portion 446 (e.g., the second side at least partially bounding the external region 414 outside the housing 410). In certain implementations, the method 500 further comprises generating the oscillating electrical voltage signals and generating the electrical stimulation signals using circuitry (e.g., circuitry 430) implanted on or within the recipient's body. For example, the circuitry can be at least partially encircled by the plurality of electrical feedthroughs.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of auditory prostheses, various implementations described herein can be incorporated in a variety of other suitable devices, methods, and contexts. More generally, as can be appreciated, certain implementations described herein can be used in a variety of implantable medical device contexts that can benefit from having an adjustable orientation of at least a portion of the implanted device during implantation.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a housing sealing an internal region within the housing from an external region outside the housing, the housing configured to be implanted on or within a recipient's body;
at least one actuator at least partially within the housing, the at least one actuator configured to generate mechanical vibrational signals, the at least one actuator comprising a coupling portion configured to be in mechanical communication with a fixture implanted on or within a recipient's body and configured to transmit the mechanical vibrational signals to the recipient's body;
circuitry within the housing and configured to generate electrical stimulation signals configured to be received by the recipient's body; and
a plurality of electrical conduits in electrical communication with the circuitry and extending from the internal region to the external region, the plurality of electrical conduits arranged in at least one arc segment at least partially encircling the coupling portion of the at least one actuator.

2. The apparatus of claim 1, further comprising a plurality of electrically conductive wires in the external region and a plurality of stimulation electrodes in the external region, each electrically conductive wire of the plurality of electrically conductive wires having a first portion in electrical communication with a corresponding electrical conduit of the plurality of electrical conduits and a second portion in electrical communication with a corresponding stimulation electrode of the plurality of stimulation electrodes, the plurality of electrically conductive wires and the plurality of stimulation electrodes configured to transmit the electrical stimulation signals from the plurality of electrical conduits to the recipient's body.

3. The apparatus of claim 2, wherein the electrical stimulation signals are configured to be received by a cochlea of the recipient's body to evoke a first hearing percept by the recipient in a first acoustic frequency range.

4. The apparatus of claim 3, wherein the mechanical vibrational signals are configured to be received by the cochlea of the recipient's body to evoke a second hearing percept by the recipient in a second acoustic frequency range, the second acoustic frequency range lower than the first acoustic frequency range.

5. The apparatus of claim 1, wherein the at least one actuator further comprises:
a substantially planar piezoelectric component within the internal region, the piezoelectric component having a first portion substantially surrounding and in mechanical communication with the coupling portion and a second portion spaced from the coupling portion, the piezoelectric component configured to undergo bending oscillations in response to received electric voltage signals; and
at least one mass within the internal region, the at least one mass in mechanical communication with the second portion and configured to move in response to the bending oscillations of the piezoelectric component.

6. The apparatus of claim 5, wherein the coupling portion extends from the fixture along a longitudinal axis, the piezoelectric component is disk-shaped and extends along a plane substantially perpendicular to the longitudinal axis, the first portion comprises an inner perimeter of a hole extending through the piezoelectric component through which the coupling portion extends, and the second portion comprises an outer perimeter of the piezoelectric component.

7. The apparatus of claim 5, wherein the at least one mass comprises a single mass having an inner perimeter substantially surrounding the coupling portion, the circuitry, and the plurality of electrical conduits.

8. The apparatus of claim 1, wherein the circuitry comprises a printed-circuit board having a hole through which the coupling portion extends.

9. The apparatus of claim 1, wherein the circuitry is further configured to control the at least one actuator.

10. The apparatus of claim 1, wherein the coupling portion is electrically conductive and is in electrical communication with the circuitry and the recipient's body, the coupling portion configured to be a reference electrode for the electrical stimulation signals.

11. A method comprising:
applying oscillating electrical voltage signals to a planar piezoelectric element having a central portion and a peripheral portion spaced from the central portion, the central portion in mechanical communication with an abutment affixed to a fixture implanted on or within a recipient's body, the peripheral portion affixed to at least one mass, the piezoelectric element responding to the electric voltage signals by imparting oscillatory motion to the at least one mass to generate vibrations and to transmit the vibrations to the fixture via the abutment; and
applying electrical stimulation signals to the recipient's body via a plurality of electrical feedthroughs substantially surrounding the abutment.

12. The method of claim 11, wherein each electrical feedthrough of the plurality of electrical feedthroughs comprises an electrically insulative portion and at least one electrical conduit extending from a first side of the electrically insulative portion to a second side of the electrically insulative portion.

13. The method of claim 11, wherein the electrical stimulation signals are configured to be received by a cochlea of the recipient's body to evoke a first hearing precept by the recipient in a first acoustic frequency range and the vibrations are configured to be received by the cochlea of the recipient's body to evoke a second hearing precept by the recipient in a second acoustic frequency range, the second acoustic frequency range lower than the first acoustic frequency range.

14. The method of claim 11, further comprising generating the oscillating electrical voltage signals and generating the electrical stimulation signals using circuitry implanted on or within the recipient's body.

15. (canceled)

16. An apparatus comprising:
an implantable stimulator comprising a plurality of signal conduits and configured to emit non-vibrational stimulation signals via the plurality of signal conduits to be received by a cochlea of the recipient's body and to evoke a first hearing percept by the recipient in a first acoustic frequency range; and
an implantable actuator comprising an abutment, the actuator configured to emit vibrational signals via the abutment, the vibrational signals configured to be received by the cochlea of the recipient's body and to evoke a second hearing percept by the recipient in a second acoustic frequency range, the second acoustic frequency range lower than the first acoustic frequency range, the abutment at least partially encircled by the plurality of signal conduits.

17. The apparatus of claim 16, wherein the plurality of signal conduits are arranged in at least one arc segment at least partially encircling the abutment.

18. The apparatus of claim 16, wherein the apparatus further comprises a plurality of feedthroughs, each feedthrough of the plurality of feedthroughs comprising an electrically insulating portion and two or more of the signal conduits extending through the electrically insulating portion.

19. (canceled)

20. The apparatus of claim 18, wherein each feedthrough of the plurality of feedthroughs has a shape of an arc segment of a circular annulus.

21. The apparatus of claim 18, wherein each feedthrough of the plurality of feedthroughs is at substantially the same distance from the abutment.

22. The apparatus of claim 16, wherein the abutment is configured to be in mechanical communication with a fixture implanted on or within a recipient's body and to transmit the vibrational signals to the recipient's body.

23. (canceled)
